(19) United States
(12) Patent Application Publication
(10) Pub. No.: US 2018/0282336 A1
(43) Pub. Date: Oct. 4, 2018

(54) Active Acrylamides

(71) Applicant: Sandoz AG, Basel (CH)

(72) Inventors: Wolfgang Felzmann, Kundl (AT); Stefanie Brunner, Kundl (AT); Hannes Lengauer, Kundl (AT)

(73) Assignee: Sandoz AG, Basel (CH)

(21) Appl. No.: 15/522,889

(22) PCT Filed: Oct. 29, 2015
(86) PCT No.: PCT/EP2015/075052

§ 371 Date: Apr. 28, 2017

(30) Foreign Application Priority Data
Oct. 30, 2014 (EP) 14191004.2

(51) Int. Cl. C07D 487/04; A61K 31/519; C07D 211/42; C07D 487/14

(52) U.S. Cl. CPC C07D 487/04; C07D 487/14; C07D 211/42; A61K 31/519

(57) ABSTRACT

The present invention refers to the synthesis and intermediates of substituted bicyclic compounds, which are used as a masked form of acrylamides and in particular refers to the synthesis of the Bruton's tyrosine kinase (Btk) inhibitor 1-((R)-3-(4-amino-3-(4-phenoxyphenyl)-1H-pyrazolo[3,4-d]pyrimidin-1-yl)piperidin-1-yl)prop-2-en-1-one (ibrutinib) and its synthesis intermediates.

### FIELD OF THE INVENTION

The present invention refers to the synthesis and intermediates of substituted bicyclic compounds, which are used as a masked form of acrylamides and in particular refers to the synthesis of the Bruton's tyrosine kinase (Btk) inhibitor 1-((R)-3-(4-amino-3-(4-phenoxyphenyl)-1H-pyrazolo[3,4-d]pyrimidin-1-yl)piperidin-1-yl)prop-2-en-1-one (ibrutinib) and its synthesis intermediates.

### BACKGROUND OF THE INVENTION

Inhibitors of kinases involved in mediating or maintaining disease states represent novel therapies for various disorders, such as hyperproliferative diseases and cancer. Bruton's tyrosine kinase (Btk), a member of the Tec family of non-receptor tyrosine kinases, is a key signaling enzyme expressed in all hematopoietic cells types except T lymphocytes and natural killer cells. Btk plays an essential role in the B-cell signaling pathway linking cell surface B-cell receptor (BCR) stimulation to downstream intracellular responses. 1-((R)-3-(4-amino-3-(4-phenoxyphenyl)-1H-pyrazolo[3,4-d]pyrimidin-1-yl)piperidin-1-yl)prop-2-en-1-one is also known by its IUPAC name as 1-{(3)-3-[4-amino-3-(4-phenoxyphenyl)-1H-pyrazolo [3,4-d]Jpyrimidin-1-yl]piperidin-1-yl}prop-2-en-1-one or 2-propen-1-one, 1-[(3R)-3-[4-amino-3-(4-phenoxyphenyl)-1H-pyrazolo[3,4-]pyrimidin-1-yl]-1-piperidinyl]-, and has been given the USAN name “Ibrutinib”, which will be used further in the document and refers to the compound with the following structure:

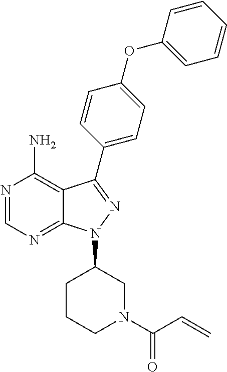 [SMILES: C=CC(=O)N1CCC[C@@H](n2nc(-c3ccc(Oc4ccccc4)cc3)c3c(N)ncnc32)C1]

Ibrutinib is an orally-administered, selective and covalent irreversible inhibitor of the enzyme Bruton's tyrosine kinase. It was first disclosed in WO 2008/039218, and has been shown to be highly clinically efficacious in relapsed/refractory chronic lymphocytic leukemia (CLL) and mantle cell lymphoma (see e.g. Burger et al., Leukemia & Lymphoma (2013), 54(11), 2385-91).

Ibrutinib has been reported to promote apoptosis, inhibit proliferation, and also prevent CLL cells from responding to survival stimuli provided by the microenvironment. Treatment of activated CLL cells with ibrutinib resulted in inhibition of Btk tyrosine phosphorylation and also effectively abrogated downstream survival pathways activated by this kinase. Additionally, ibrutinib inhibited proliferation of CLL cells in vitro, effectively blocking survival signals provided externally to CLL cells from the microenvironment. Further, ibrutinib has been reported to inhibit cellular adhesion following stimulation at the B cell receptor. Together, these data are consistent with a mechanistic model whereby ibrutinib blocks B cell receptor signaling, which drives cells into apoptosis and/or disrupts cell migration and adherence to protective tumour microenvironments.

WO 01/019829 describes a general synthesis for substituted 1H-pyrazolo[3,4-d] pyrimidines. A Knoevenagel-condensation of phenoxybenzoic acid chloride and malonic acid dinitrile furnishes the enole, which is subsequently methylated using hazardous TMS diazomethane. The pyrazole- and pyrimidine ring systems are then assembled via two successive condensation reactions.

WO 2008/039218 and WO2008/121742 describe a synthesis of Ibrutinib, with the 1H-pyrazolo[3,4-d] pyrimidine being assembled according to WO 0119829 A2. The coupling of the chiral piperidine building block is accomplished via a Mitsunobu reaction, generating a large waste stream. Ibrutinib is then obtained after a final protecting group manipulation (Boc-removal followed by coupling with acryloyl chloride). In total, the described process comprises an uneconomical high number of eight process steps, furthermore compound Va is produced as a by-product in the last step.

In CN 103121999 a 1H-pyrazolo[3,4-d] pyrimidine is obtained via palladium-catalyzed cross-coupling of a 3-Halo-1H-pyrazolo[3,4-d] pyrimidine with phenoxyphenyl boronic acid—both of which being very expensive chemicals. In contrast to WO08039218, an additional trifluoroacetyl is introduced which has to be removed at the end of the synthetic sequence.

CN 103626774 discloses a synthesis starting with a Knoevenagel-condensation of phenoxybenzoic acid chloride and malonic acid dinitrile, furnishing an enol-ether after methylation with dimethyl sulphate. The pyrazole ring system is assembled via condensation with a piperidinyl hydrazine. A final condensation reaction then gives rise to Ibrutinib. WO2014/139970 describes a similar sequence, with emphasis on the synthesis of the complex piperidinyl hydrazine derivatives used for the pyrazole synthesis. However, the preparation of the chiral piperidinyl hydrazine derivative requires a costly chiral chromatography step. Furthermore, the final step has the same drawbacks as described in WO2008/039218.

In view of the above described prior art, a need exists for a more efficient synthetic route for the synthesis of substituted 1H-pyrazolo[3,4-d] pyrimidines, such as ibrutinib and derivatives thereof. In particular, the synthesis should be more economical then the synthetic routes of the prior art, i.e. should need only a reduced number of process steps, and which can start from cheap materials. Further, a synthesis free from use or generation of hazardous materials is desired. In particular, it should avoid the generation of large waste streams, for example by avoiding an uneconomical Mitsunobu reaction. It is therefore desired to find a new synthesis for ibrutinib and its derivatives, which overcomes the disadvantages of the prior art processes.

Further, a need exists in the art for the synthesis of novel substituted 1H-pyrazolo[3,4-d]pyrimidines to find novel therapeutic agents active as receptor or non-receptor tyrosine kinase inhibitors, in particular, Btk inhibitors.

It is has surprisingly been found in the present invention that the problems of the prior art can be solved by the provision of a synthesis for substituted 1H-pyrazolo[3,4-d] pyrimidines, such as ibrutinib and derivatives thereof, which uses a protected form of acrylamides, i.e. active acrylamides. Acrylamides are reactive structural elements, and can undergo uncontrolled polymerization under both acidic and basic conditions. Therefore, such structural elements are normally introduced into the molecule as late as possible. However, it has surprisingly been found in the present invention that by using a protected (masked) form of acrylamide, it can be introduced earlier and in a more convergent manner, leaving the deprotection (de-masking) as the final step.

### DESCRIPTION OF THE INVENTION

An “alkyl” group refers to a hydrocarbon group, which is not aromatic. The alkyl moiety may be a “saturated alkyl” group, which means that it does not contain any carbon-carbon double or triple bonds. The alkyl moiety may also be an “unsaturated alkyl” moiety, which means that it contains at least one carbon-carbon double or triple bond. “Unsaturated alkyl” moieties containing at least one carbon-carbon double bond are referred to as an “alkene” moiety. “Unsaturated alkyl” moieties containing at least one carbon-carbon triple bond are referred to as an “alkyne” moiety. The alkyl moiety, whether saturated or unsaturated, may be branched or straight chain.

The (saturated) “alkyl” group may have 1 to 10 carbon atoms (whenever it appears herein, a numerical range such as “1 to 10” refers to each integer in the given range; e.g., “1 to 10 carbon atoms” means that the alkyl group may have 1 carbon atom, 2 carbon atoms, 3 carbon atoms, etc., up to and including 10 carbon atoms, although the present definition also covers the occurrence of the term “alkyl” where no numerical range is designated). The alkyl group of the compounds described herein may be designated as “C₁-C₄ alkyl” or similar designations. By way of example only, “C₁-C₄ alkyl” indicates that there are one to four carbon atoms in the alkyl chain, i.e., the alkyl chain is selected from among methyl, ethyl, propyl, isopropyl, n-butyl, isobutyl, sec-butyl, and t-butyl. Typical alkyl groups include, but are not limited to, methyl, ethyl, propyl, isopropyl, butyl, isobutyl, tertiary butyl, pentyl, 2-methylbutyl, 3-methylbutyl, 3,3-dimethylpropyl, hexyl, 2-methylpentyl, 3,3-dimethylbutyl, 2, 3-dimethyl butyl and the like. Alkyl groups can be substituted or unsubstituted. For example, alkyl groups may be substituted by aromatic groups, such as phenyl-substituted methyl, i.e. benzyl.

As indicated above the term “alkenyl” refers to a type of unsaturated alkyl group that is not part of an aromatic group. Alkenyl groups may have 2 to 10 carbons. The alkenyl moiety may be branched or straight chain. Alkenyl groups can be optionally substituted. Non-limiting examples of an alkenyl group include —C(CH₃)═CH₂, —CH═CH₂, —CH═C(CH₂CH₃)₂, —CH═CHCH₃, —C(CH₃)═CHCH₃. As indicated above the term “alkynyl” refers to a type of unsaturated alkyl group in which two atoms of the alkyl group form a triple bond. Alkynyl groups may have 2 to 10 carbons. The alkynyl moiety may be branched or straight chain. Alkynyl groups can be optionally substituted. Non-limiting examples of an alkynyl group include, but are not limited to, —C═CH, —C═CCH₃, —C≡CCH₂CH₃.

A heteroalkyl group refers to an alkyl group as defined above wherein at least one carbon atom is substituted with a heteroatom such as nitrogen, oxygen, sulphur and/or phosphorus.

A “cycloalkyl” group refers to a hydrocarbon group, which is not aromatic and wherein at least three carbon atoms are forming a ring. As used herein, the term “ring” refers to any covalently closed structure. Rings can be monocyclic or polycyclic. The cycloalkyl moiety may be a “saturated cycloalkyl” group, which means that it does not contain any carbon-carbon double or triple bonds. The cycloalkyl moiety may also be an “unsaturated cycloalkyl” moiety, which means that it contains at least one carbon-carbon double or triple bond. The (saturated) “cycloalkyl” moiety may have 3 to 12 carbon atoms. The cycloalkyl group of the compounds described herein may be designated as “C₃-C₁₂ cycloalkyl” or similar designations. By way of example only, “C₃-C₅ cycloalkyl” indicates that there are three to five carbon atoms in the cycloalkyl ring, i.e. the cycloalkyl ring is selected from among cyclopropyl, cyclobutyl, and cyclopentyl. Typical cycloalkyl groups include, but are in no way limited to cyclopropyl, cyclobutyl, and cyclopentyl, cyclohexyl, cycloheptyl, cyclooctyl and the like. Cycloalkyl groups can be substituted or unsubstituted.

As indicated above a “cycloalkenyl” group refers to an unsaturated cycloalkyl group, wherein at least five carbon atoms are forming a ring. The “cycloalkenyl” moiety may have 5 to 12 carbon atoms. The cycloalkenyl group of the compounds described herein may be designated as “C₅-C₁₂ cycloalkenyl” or similar designations. By way of example only, “C₅-C₈ cycloalkenyl” indicates that there are five to eight carbon atoms. Cycloalkenyl groups can be substituted or unsubstituted. Typical cycloalkenyl groups include, but are in no way limited to cyclopentenyl, cyclohexenyl, cycloheptenyl, cyclooctenyl and the like.

A heterocycloalkyl group refers to a cycloalkyl group as defined above wherein at least one carbon atom being part of the ring is a heteroatom such as nitrogen, oxygen sulphur and/or phosphorus.

The term “aryl” group refers to a residue with an aromatic skeletal structure, wherein the ring atoms of the aromatic skeletal structure are carbon atoms. The term “aromatic” refers to a planar ring having a delocalized [pi]-electron system containing 4n+2 [pi] electrons, where n is an integer. The aryl group can be formed from five, six, seven, eight, nine, or more than nine atoms. Aryl groups can be optionally substituted. The aryl groups can be monocyclic or polycyclic (i.e., rings which share adjacent pairs of carbon atoms) groups.

Examples of aryl groups include, but are not limited to phenyl, biphenyl, naphthyl, binaphthyl, pyrenyl, azulenyl, phenanthryl, anthracenyl, fluorenyl, and indenyl.

The term heteroaryl group refers to an aryl group as defined above wherein at least one carbon atom being part of the aromatic skeletal ring structure is a heteroatom such as nitrogen, oxygen, sulphur and/or phosphorus.

Examples of heteroaryl groups include, but are not limited to pyrrolyl, imidazolyl, furyl, thienyl, oxazolyl, thiazolyl, thiadiazolyl, tetrazolyl, pyridyl, pyrimidyl, triazolyl, indolyl, isoindolyl, benzofuranyl, dibenzofuranyl, benzothienyl, benzimidazolyl.

The above (hetero)alkyl, (hetero)cycloalkyl and (hetero)aryl groups can optionally be substituted with one or more substituents. Examples of substituents are alkyl, heteroalkyl, cycloalkyl, heterocycloalkyl, aryl, heteroaryl, alkoxy, cycloalkoxy, aryloxy, alkylthio, cycloalkylthio, arylthio, alkylsulfoxide, arylsulfoxide, alkylsulfone and arylsulfone.

Further Examples of substituents are cyano, nitro, halogen, hydroxy or protected hydroxy groups, amines or protected amines, monoalkyl amines or protected monoalkyl amines, monoarylamines or protected monoarylamines, dialkylamines, diarylamines, amides and esters.

An “amide” is a chemical moiety is with the functional group —C(O)NR₂, where R refers to H or organic groups, and preferably refers to a chemical moiety with the formula —C(O)NHR or —NHC(O)R^{A}, where R^{A} may be selected from mong (hetero)alkyl, (hetero)aryl and (hetero)cycloalkyl as described herein.

The term “ester” refers to a chemical moiety with formula —COOR^{E}, where R^{E} is selected from among (hetero)alkyl, (hetero)cycloalkyl and (hetero)aryl groups as described herein.

The term “halogen” comprises chloro, bromo and iodo.

The term “monoalkylamine” refers to the —NH(alkyl), where the alkyl groups are as defined herein.

The term “dialkylamine” refers to the —N(alkyl)₂, where the alkyl groups are as defined herein or further when taken together with the N atom to which they are attached, can optionally form a cyclic ring system.

The term “diarylamine” refers to the —N(aryl)₂, where the aryl groups are as defined herein. Protection groups for amines or mono-substituted amines are for example Boc (tert-butyloxycarbonyl), Z or Cbz (benzyloxycarbonyl), benzyl, benzhydryl and Fmoc (fluorenylmethylenoxycarbonyl).

Protection groups for hydroxyl groups are for example esters, such as benzoic acid esters or pivalic acid esters, and trisubstituted silylethers, such as trimethylsilylether, triethylsilylether, tert-butyldimethylsilylether and tert-butyl diphenylsilylether.

Further examples of suitable amine or hydroxyl protecting groups can be found in Greene, P. G. M.; Wuts, T. W. Greene's Protective Groups in Organic Synthesis, 4^{th} Edition, 2007, John Wiley & Sons, Hoboken, N.J.

### DETAILED DESCRIPTION OF EMBODIMENTS OF THE INVENTION

In a first embodiment, the present invention refers to a process for the preparation of a compound of formula (IV),

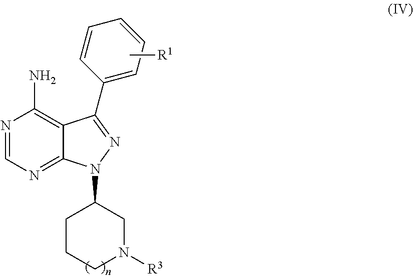

comprising the steps of:

(a) reacting a compound of formula (II)

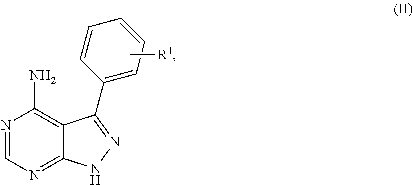

in the presence of an alkaline substance, and optionally in the presence of a phase transfer catalyst, with a compound of formula (III)

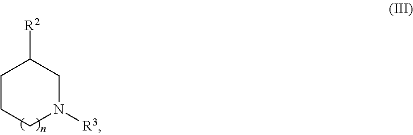

to obtain a compound of formula (IV),
wherein R¹ is selected from substituted or non-substituted alkyl, substituted or non-substituted heteroalkyl, substituted or non-substituted cycloalkyl, substituted or non-substituted heterocycloalkyl, OR⁴, SR⁴, NR⁴R⁵, and halogen, preferably is OR⁴.
R⁴ and R⁵ are individually selected from hydrogen, substituted or non-substituted alkyl, substituted or non-substituted heteroalkyl, substituted or non-substituted cycloalkyl, substituted or non-substituted heterocycloalkyl, substituted or non-substituted aryl, and substituted or non-substituted heteroaryl, preferably aryl, most preferably phenyl,
n is 0 to 3, preferably is 1,
R² is a leaving group,
R³ is selected from hydrogen, a group selected from carbamoyl, carbamates of the formula C(O)O—R⁹, substituted or non-substituted benzyl and substituted or non-substituted silyl, and C(O)—R⁶, wherein
R⁶ is selected from hydrogen, substituted or non-substituted alkyl, substituted or non-substituted heteroalkyl, substituted or non-substituted cycloalkyl, substituted or non-substituted heterocycloalkyl, substituted or non-substituted alkenyl, substituted or non-substituted cycloalkenyl, substituted or non-substituted heterocycloalkenyl, substituted or non-substituted aryl, and substituted or non-substituted heteroaryl, and
R⁹ is selected from substituted or non-substituted alkyl, substituted or non-substituted heteroalkyl, substituted or non-substituted cycloalkyl, substituted or non-substituted heterocycloalkyl, substituted or non-substituted alkenyl, substituted or non-substituted cycloalkenyl, substituted or non-substituted heterocycloalkenyl substituted or non-substituted aryl, and substituted or non-substituted heteroaryl.

The process may be represented by the following reaction scheme:

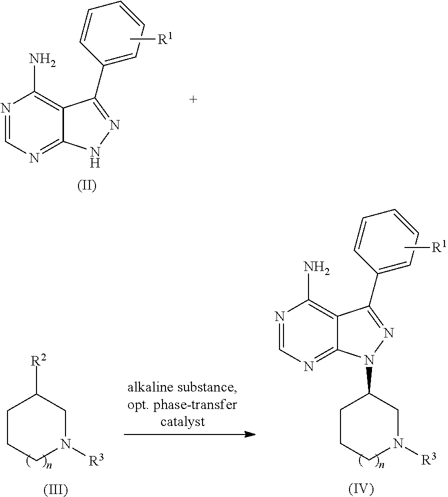

wherein n, R¹, R² and R³ are as defined above.

The leaving group R² is not particularly restricted, but may be any molecular fragment that departs with a pair of electrons in heterolytic bond cleavage. Preferably, the leaving group R² is selected from halogens, such as Cl, Br and I, or appropriately functionalised esters, such as carboxylic acid esters, sulfates, sulfonates, tosylates, mesylates or phosphates. More preferred leaving groups R² are bromides, monochloromesylates (monochlates) and mesylates, most preferably is mesylate.

Preferably, the compound of formula (III) can be in the form of its (S)-enantiomer, i.e. R² is arranged such that the corresponding (S) configuration is obtained.

In a preferred embodiment, R³ is C(O)—R⁶, and R⁶ is preferably substituted or non-substituted (hetero)cycloalkenyl, and most preferably a group selected from one of the following

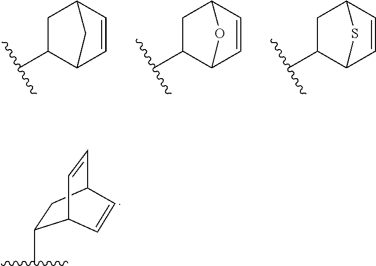 [SMILES: *C1CC2C=CC1C2 ; *C1CC2C=CC1O2 ; *C1CC2C=CC1S2 ; *C1CC2C=CC1C=C2]

The phase transfer catalyst used in the reaction described above is typically a compound of the formula

NR⁸₄⁺X⁻ or PR⁸₄⁺X⁻
wherein R⁸ is individually selected from substituted or non-substituted alkyl, substituted or non-substituted heteroalkyl, substituted or non-substituted cycloalkyl, substituted or non-substituted heterocycloalkyl, substituted or unsubstituted aryl or substituted or unsubstituted heteroaryl and
X is selected from Cl, Br, F, SbF₆, PF₆, BF₄, ClO₄, HSO₄, HCO₃, NO₃, CF₃COO, alkyl-COO, aryl-COO, alkyl-SO₃, aryl-SO₃, and CF₃SO₃.

The ammonium salts are preferred to the phosphonium salts for the purposes of the present invention. Particularly preferred are tetran-butyl ammonium, tri-n-butyl methyl ammonium and benzyl triethyl ammonium salts with X=chloride, bromide and hydrogen sulfate.

A most particularly preferred phase transfer catalyst is ALIQUAT® HTA-I (manufacturer: Cognis/BASF). Another phase transfer catalyst preferred for the purposes of the present invention is ALIQUAT® 175 (manufacturer: Cognis/BASF). Another phase transfer catalyst preferred for the purposes of the present invention is tetrabutyl ammoniumbromide (TBAB). The phase transfer catalysts are typically used in catalytic quantities. The quantity in which the phase transfer catalysts are used can vary between 0.1 and 25 mol %, based on the amount of reactants, and is preferably between 1 and 10 mol % and more particularly between 5 and 8 mol %.

In a further preferred embodiment, the compound of formula (III) is a compound having the formula (IIIa)

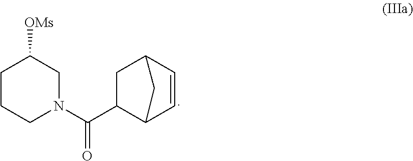 [SMILES: CS(=O)(=O)O[C@H]1CCCN(C(=O)C2CC3C=CC2C3)C1]

The alkaline substance preferably comprises one or more of NaH, KH, NaNH₂, sodium ethoxide and potassium t-butoxide, amide bases including Natrium-bis(trimethylsilyl)amid and Lithiumdiisopropylamid, K₂CO₃ and Cs₂CO₃. Most preferably, the alkaline substance is K₂CO₃ in the presence of a phase-transfer catalyst. The base material, such as K₂CO₃, may be added to the reaction mixture as aqueous solution, aqueous dispersion or in solid form.

In a particular preferred embodiment of the above described process of the present invention,
R¹ is OR⁴ and R⁴ is phenyl,
n is 1, and
R³ is C(O)—R⁶, and R⁶ is

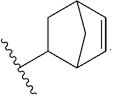 [SMILES: *C1CC2C=CC1C2]

In this particular preferred embodiment, the compound of formula (IV) is the compound of formula (IVa)

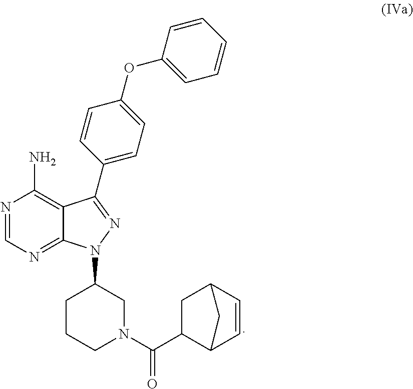 [SMILES: Nc1ncnc2c1c(-c1ccc(Oc3ccccc3)cc1)nn2[C@@H]1CCCN(C(=O)C2CC3C=CC2C3)C1]

In a typical example, the reaction is carried out using 1 to 3 eq. of compound Illa relative to compound II.

Suitable solvents for the above described process, without being limited to, include anisole, methyl-tetrahydrofuran, toluene, xylene and mesitylene, methyl ethyl ketone, methyl isobutyl ketone, which may be used in the presence of an aqueous solution of base, preferably a saturated solution of K₂CO₃.

The reaction is typically carried out at elevated temperature, preferably 80° C. to 180° C., even more preferably 100° C. to 160° C., further preferably 110° C. to 140° C. After completed reaction, the product may be isolated by precipitation as a salt, preferably a nitrate, the salt of 1,5-naphthyldisulfonic acid, a monohydrochloride salt or dihydrochloride salt, which are prepared by the addition of suitable amounts of the respective acids. For some embodiments, specific salts such as the monohydrochloride are preferred for the purpose of enantiomeric purification.

In a further embodiment, the invention refers to a process for the preparation of a compound of formula (I),

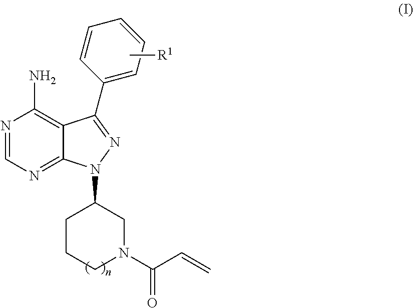

by subjecting a compound of formula (IV)

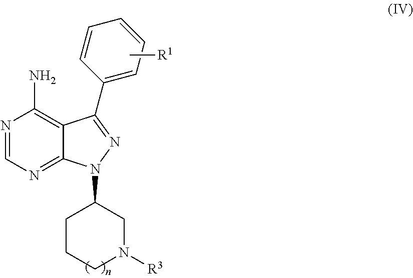

to deprotection to obtain the compound of formula (I),
wherein R is selected from substituted or non-substituted alkyl, substituted or non-substituted heteroalkyl, substituted or non-substituted cycloalkyl, substituted or non-substituted heterocycloalkyl, OR⁴, S, NR⁴R⁵, and halogen,
R⁴ and R⁵ are individually selected from hydrogen, substituted or non-substituted alkyl, substituted or non-substituted heteroalkyl, substituted or non-substituted cycloalkyl, substituted or non-substituted heterocycloalkyl, substituted or non-substituted aryl, and substituted or non-substituted heteroaryl,
n is 0V to 3,
R³ is C(O)—R⁶, and
R⁶ is a group selected from

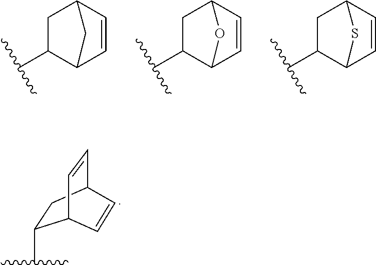 [SMILES: *C1CC2C=CC1C2 ; *C1CC2C=CC1O2 ; *C1CC2C=CC1S2 ; *C1CC2C=CC1C=C2]

The process may be represented by the following reaction scheme:

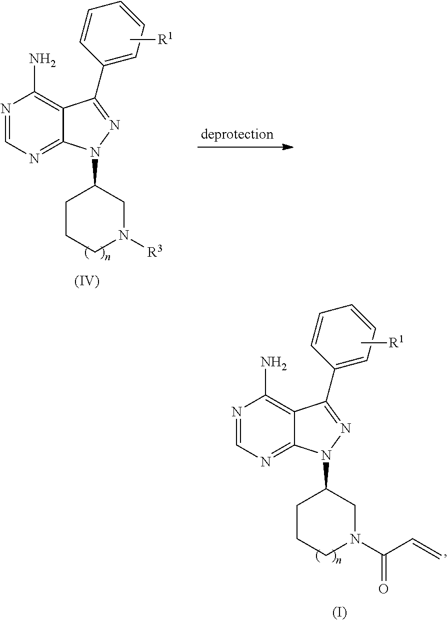

wherein R¹, R³ and n are as described above.

Typically, the compound of formula (IV) is obtained by the process as described further above.

Deprotection is typically performed at 180° C. to 280° C., preferably at 210° C. to 260° C.

Further, deprotection is typically performed in a solvent with a boiling point above 150° C., and can be selected from one or more of diphenyl ether, sulfolane, dimethylsulfoxide, dimehthylacetamide, N-methyl pyrrolidinone, mesitylene, anisole, ethylene glycol butyl ether or di-, tri and tetraglyme. Preferably the reaction is carried out in diphenyl ether. Additionally, stabilisers like butylated hydroxytoluene (2,6-di-tert-butyl-4-methylphenol, BHT) or scavengers like maleic anhydride can be present.

In a preferred embodiment, n is 1, R³ is C(O)—R⁶, and R⁶ is

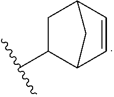 [SMILES: *C1CC2C=CC1C2]

The process of this preferred embodiment may be represented by the following reaction scheme:

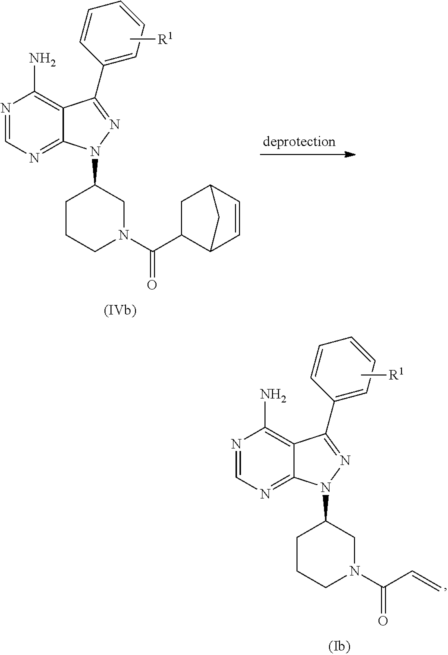

wherein R¹ is as described above.

In a further preferred embodiment, R¹ is OPhenyl, n is 1, R³ is C(O)—R⁶, and R⁶ is

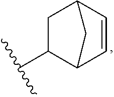 [SMILES: *C1CC2C=CC1C2]

the process resulting in a compound of formula (Ia)

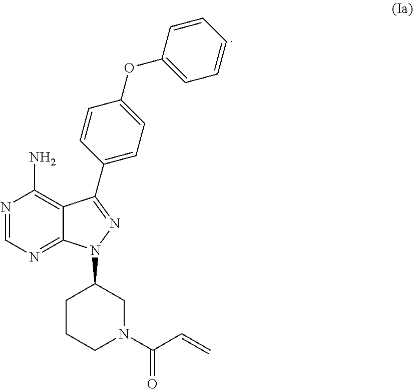 [SMILES: C=CC(=O)N1CCC[C@@H](n2nc(-c3ccc(Oc4ccccc4)cc3)c3c(N)ncnc32)C1]

The process of this preferred embodiment may be represented by the following reaction scheme:

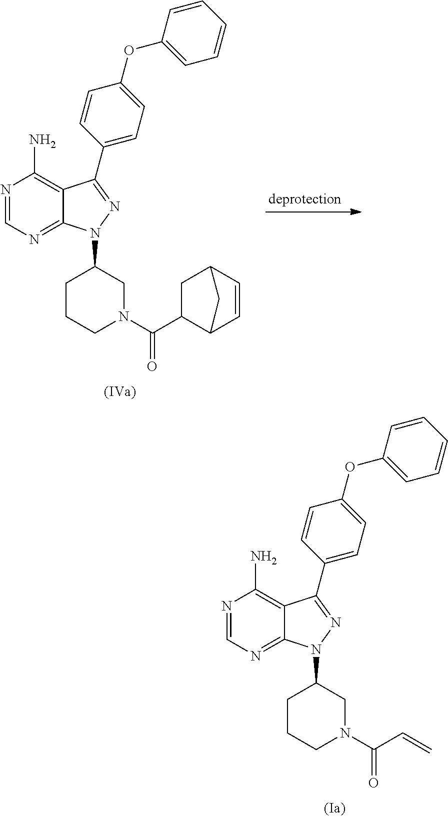 [SMILES: Nc1ncnc2c1c(-c1ccc(Oc3ccccc3)cc1)nn2[C@@H]1CCCN(C(=O)C2CC3C=CC2C3)C1 ; C=CC(=O)N1CCC[C@@H](n2nc(-c3ccc(Oc4ccccc4)cc3)c3c(N)ncnc32)C1]

wherein deprotection is typically performed as described above. After deprotection the reaction product may be isolated by crystallisation.

In another embodiment, the present invention refers to a process for the preparation of a compound of formula (Ia),

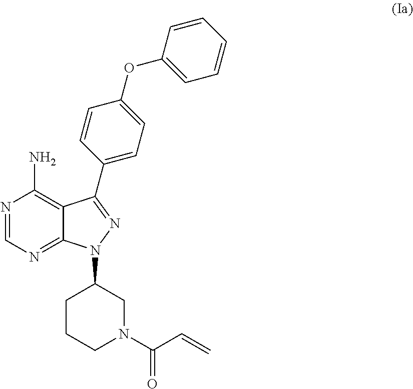 [SMILES: C=CC(=O)N1CCC[C@@H](n2nc(-c3ccc(Oc4ccccc4)cc3)c3c(N)ncnc32)C1]

comprising the process steps of:

(a) reacting a compound of formula (IIa)

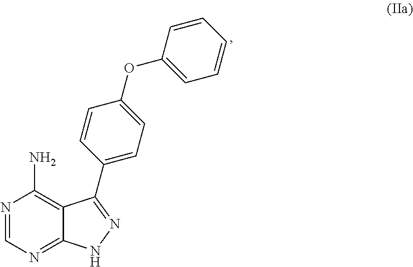 [SMILES: Nc1ncnc2[nH]nc(-c3ccc(Oc4ccccc4)cc3)c12]

in the presence of an alkaline substance, and optionally in the presence of a phase transfer catalyst, with a compound of formula (IIIa)

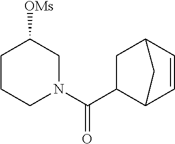 [SMILES: CS(=O)(=O)O[C@H]1CCCN(C(=O)C2CC3C=CC2C3)C1]

to obtain a compound of formula (IVa),

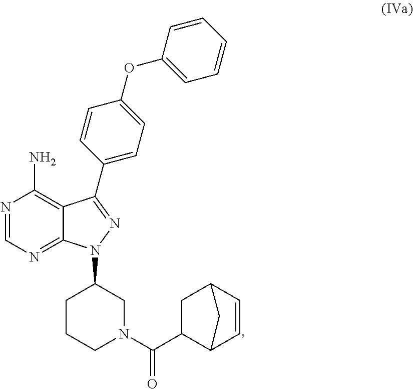 [SMILES: Nc1ncnc2c1c(-c1ccc(Oc3ccccc3)cc1)nn2[C@@H]1CCCN(C(=O)C2CC3C=CC2C3)C1]

and subjecting the compound of formula (IVa) to deprotection to obtain the compound of formula (Ia). In this embodiment, the preferred reagents and reaction conditions are as described above.

The process of this embodiment may be represented by the following reaction scheme:

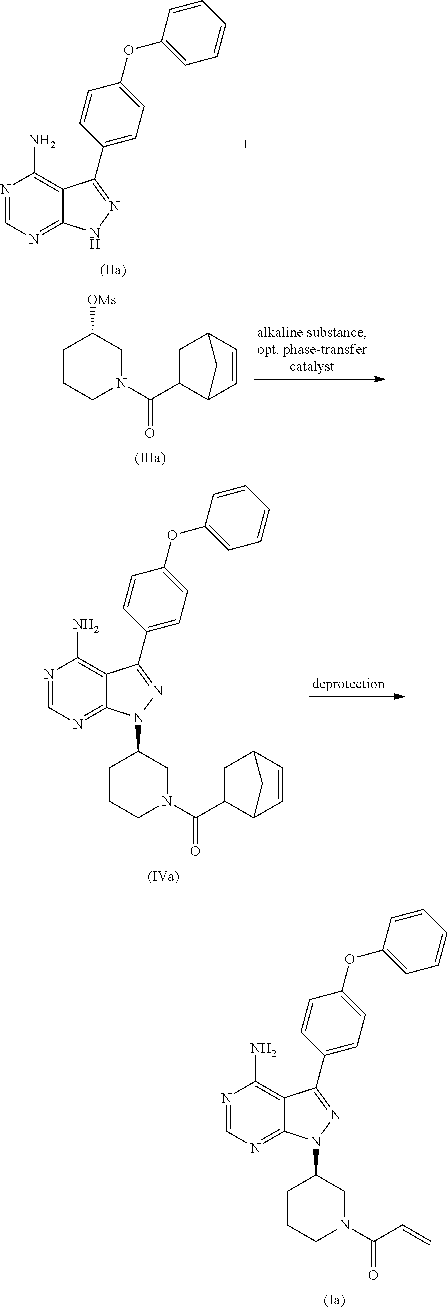 [SMILES: Nc1ncnc2[nH]nc(-c3ccc(Oc4ccccc4)cc3)c12 ; CS(=O)(=O)O[C@H]1CCCN(C(=O)C2CC3C=CC2C3)C1 ; Nc1ncnc2c1c(-c1ccc(Oc3ccccc3)cc1)nn2[C@@H]1CCCN(C(=O)C2CC3C=CC2C3)C1 ; C=CC(=O)N1CCC[C@@H](n2nc(-c3ccc(Oc4ccccc4)cc3)c3c(N)ncnc32)C1]

In another embodiment, the present invention refers to a process for the preparation of a compound of formula (VIII),

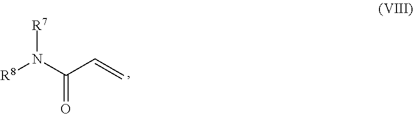

comprising the steps of:

subjecting a compound of formula (VII)

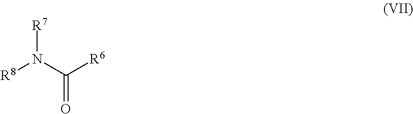

to deprotection,
wherein R⁶ is a group selected from one of the following moieties

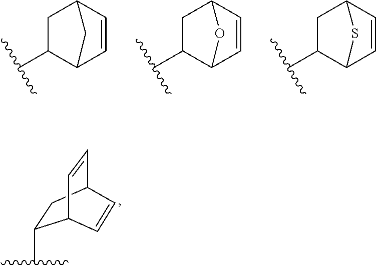 [SMILES: *C1CC2C=CC1C2 ; *C1CC2C=CC1O2 ; *C1CC2C=CC1S2 ; *C1CC2C=CC1C=C2]

and
R⁷ and R⁸ are individually selected from hydrogen, substituted or non-substituted alkyl, substituted or non-substituted heteroalkyl, substituted or non-substituted cycloalkyl, substituted or non-substituted heterocycloalkyl, substituted or non-substituted aryl, and substituted or non-substituted heteroaryl.

Preferably, R⁷ and R⁸ together form a substituted or non-substituted heterocycloalkyl. Most preferably, R⁷ and R⁸ together form (S)-3-(4-phenoxyphenyl)-1-(piperidin-3-yl)-1H-pyrazolo[3,4-d]pyrimidin-4-amine. Deprotection is typically performed at 180° C. to 280° C., preferably at 210° C. to 260° C.

Further, deprotection is typically performed in a solvent with a boiling point above 150° C., and can be selected from one or more of diphenyl ether, sulfolane, dimethylsulfoxide, dimethylacetamide, N-methyl pyrrolidinone, mesitylene, anisole, ethylene glycol butyl ether or di-, tri and tetraglyme. Preferably the reaction is carried out in diphenyl ether. Additionally, stabilisers like butylated hydroxytoluene (2,6-di-tert-butyl-4-methylphenol, BHT) or scavengers like maleic anhydride can be present.

The process may be represented by the following reaction scheme:

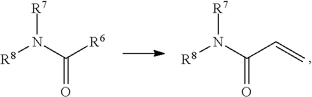

wherein R⁶, R⁷ and R⁸ are as defined above.

In a further embodiment, the present invention refers to a composition comprising a compound of formula (Ia) and a compound of formula (Va)

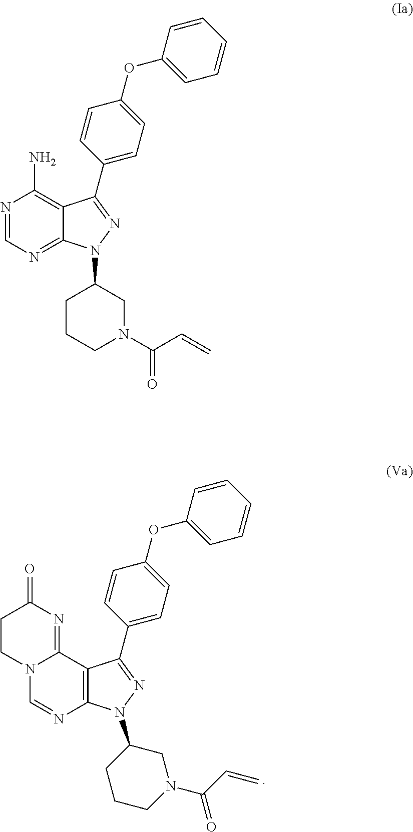 [SMILES: C=CC(=O)N1CCC[C@@H](n2nc(-c3ccc(Oc4ccccc4)cc3)c3c(N)ncnc32)C1 ; C=CC(=O)N1CCC[C@@H](n2nc(-c3ccc(Oc4ccccc4)cc3)c3c2N=CN2CCC(=O)N=C32)C1]

Preferably, the content of the compound of formula (Va) is 0.5 wt. % or less, further preferably is 0.05 wt. % or less, most preferably 0.005 wt. % or less, based on the total weight of the compounds of formula (Ia) and (Va).

The composition comprising a compound of formula (Ia) and a compound of formula (Va) is typically present as physical mixture, dispersion, aqueous solution or solution in an organic solvent.

In a further embodiment, the present invention refers to a compound represented by the formula (IIIa)

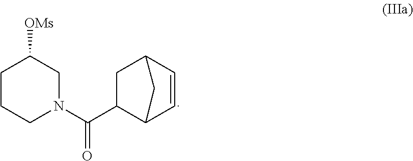 [SMILES: CS(=O)(=O)O[C@H]1CCCN(C(=O)C2CC3C=CC2C3)C1]

In particular, the present invention refers to the use of a compound represented by the formula (IIIa) in a method for preparing ibrutinib or a derivative thereof.

In a further embodiment, the present invention refers to a compound represented by the formula (IVc)

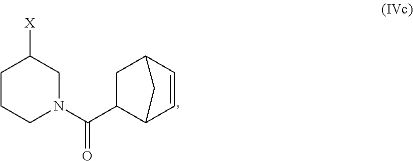

wherein X is a N-linked heterocycle. The heterocycle is preferably a substituted or unsubstituted pyrazolo[3,4-d]pyrimidine, more preferably a substituted or unsubstituted 4-aminopyrazolo[3,4-d]pyrimidine, most preferably 4-amino-3-aryl-pyrazolo[3,4-d]pyrimidine.

In particular, the present invention refers to the use of a compound represented by the formula (IVc) in a method for preparing ibrutinib or a derivative thereof.

In a preferred embodiment, the compound of formula (IVc) is represented by the formula (IVb)

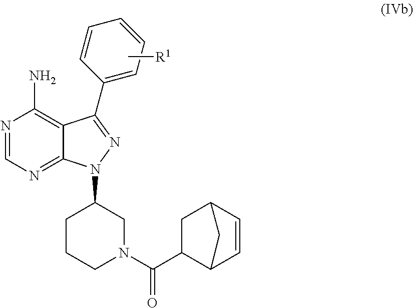

wherein R¹ is selected from substituted or non-substituted alkyl, substituted or non-substituted heteroalkyl, substituted or non-substituted cycloalkyl, substituted or non-substituted heterocycloalkyl, OR⁴, SR⁴, NR⁴R⁵, and halogen, and
R⁴ and R⁵ are individually selected from hydrogen, substituted or non-substituted alkyl, substituted or non-substituted heteroalkyl, substituted or non-substituted cycloalkyl, substituted or non-substituted heterocycloalkyl, substituted or non-substituted aryl, and substituted or non-substituted heteroaryl.

Most preferably, R¹ is OR⁴ and R⁴ is phenyl.

In a further embodiment, the present invention refers to a compound represented by the formula (Vb),

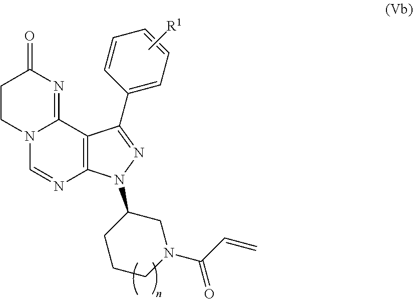

wherein n is 0 to 3, preferably is 1, and R¹ is preferably OPhenyl, as described above.

The synthetic route of the present invention for the synthesis of substituted 1H-pyrazolo[3,4-d] pyrimidines, and in particular for the synthesis of ibrutinib and derivatives thereof comprises fewer synthetic steps as the prior art processes, thus is more convergent and more efficient, and in particular avoids an uneconomical Mitsunobu reaction. Moreover, no hazardous reagents such as TMS diazomethane are required. Moreover, it starts from significantly cheaper materials. Further, it employs less protecting group manipulations, is free of phosphines, or transition metal mediated couplings, which may contaminate the active ingredient. The described installation of the acrylamide moiety completely disables the formation of by-product V which in turn leads to a cleaner API. Moreover, it is more economical than the prior art syntheses, as it eliminates the generation of hazardous materials and large waste streams, for example, no toxic acrylate reagents are used in the final synthesis step, thereby leading to the efficient synthesis of ibrutinib and derivatives thereof.

Further particular, the process of the present invention can efficiently deplete quaternary ammonium salts used as phase-transfer catalysts, which may otherwise be present in the final product as impurity. Further, the final active acrylamide compound can be liberated under neutral conditions avoiding any basic or acidic conditions which can lead to the formation of degradation- or by-products. Further, the synthesis as described herein allows modular access to substituted N-alkyl pyrazolo pyrimidines, in turn enabling library synthesis for new drug identification.

Dosage Form

Ibrutinib or any of the substituted 1H-pyrazolo[3,4-d] pyrimidines prepared by the above-described processes may be used for the manufacture of a pharmaceutical composition. Thus, in a further embodiment, the present invention relates to a pharmaceutical composition comprising a compound prepared by the processes as described herein, and in particular relates to a pharmaceutical composition comprising ibrutinib or one of its derivatives as prepared by a process as described herein.

The pharmaceutical composition typically comprises 1.0 to 1000 mg, preferably comprises 10 to 800 mg, most preferably comprises 50 to 550 mg of the compounds prepared by the above-described processes, such as ibrutinib, particularly amorphous ibrutinib.

The pharmaceutical composition may further comprise one or more pharmaceutically acceptable additives, such as binders, carriers, stabilizers, diluents, dispersing agents, suspending agents, thickening agents, and/or excipients. Suitable excipients include, for example, fillers such as sugars, including lactose, sucrose, mannitol, or sorbitol; cellulose preparations such as, for example, maize starch, wheat starch, rice starch, potato starch, gelatine, gum tragacanth, methylcellulose, micro crystalline cellulose, hydroxypropylmethylcellulose, sodium carboxymethylcellulose; or others such as: polyvinylpyrrolidone (PVP or povidone) or calcium phosphate. If desired, disintegrating agents may be added, such as the cross-linked croscarmellose sodium, polyvinylpyrrolidone, agar, or alginic acid or a salt thereof such as sodium alginate.

The pharmaceutical composition facilitates administration of the compound to a mammal, preferably to a human. Ibrutinib can be used singly or in combination with one or more therapeutic agents as components of mixtures.

The pharmaceutical composition is typically a solid oral dosage forms. It may be administered to a subject by multiple administration routes, including but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations. Preferably, the pharmaceutical dosage form is a tablet or capsule.

The pharmaceutical compositions may be manufactured in a conventional manner, such as, by way of example only, by means of conventional mixing, dissolving, granulating, dragee-making, levigating, emulsifying, encapsulating, entrapping or compression processes.

In another embodiment, the compounds prepared by the above-described processes, such as ibrutinib or a derivative thereof, are used in the treatment of cancer. In particular, cancer may be a B cell malignancy, preferably selected from chronic lymphocytic leukemia (CLL)/small lymphocytic lymphoma (SLL), mantle cell lymphoma (MCL), indolent non-Hodgkins's lymphoma, diffuse large B Cell lymphoma (DLBCL), multiple myeloma(MM), marginal zone lymphoma (NHL), hairy cell leukemia, acute lymphocyte leukemia (ALL), and breast cancer.

### EXAMPLES

In the following, the present invention will further be described by the way of non-limiting examples.

### Example 1: (3S)-1-((2R)-bicyclo[2.2.1]hept-5-ene-2-carbonyl)piperidin-3-yl methane-sulfonate

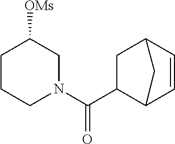 [SMILES: CS(=O)(=O)O[C@H]1CCCN(C(=O)C2CC3C=CC2C3)C1]

3.52 g (R)-3-hydroxypiperidine hydrochloride (25.58 mmol, 1.2 eq.), 8.17 g 1-ethyl-3-(3-dimethylaminopropyl)carbodiimid hydrochloride (EDC/HCl) (42.63 mmol, 2 eq.) and 2.88 g 1-hydroxybenzotriazol (HOBt) (21.32 mmol, 1 eq) were dissolved in 30 mL dimethylformamide (DMF). Then 8.9 mL triethylamine (6.47 g, 63.95 mmol, 3 eq.) were added, followed by 2.95 g 5-norbornene-2-carboxylic acid (21.32 mmol, 1 eq.). The reaction was stirred for 24 h.

The reaction mixture was then diluted with 100 mL ethyl acetate and then washed subsequently with 0.5M HCl, 0.5M NaOH and brine, dried over Na₂SO₄, filtered and concentrated under reduced pressure to give 2.63 g of crude product.

¹H NMR (500 MHz, DMSO-d₆): 6.14 (m, 2H), 4.71 (m, 1H), 3.83-3.26 (m, 5H), [3.24, 3.23, 3.20, 3.19 (s, 3H, OMs)], 2.87 (m, 1H), 2.35 (m, 1H), 1.97 (m, 1H), 1.88-1.21 (m, 7H) The residue was then dissolved in 25 mL dichloromethane (DCM) and cooled to 0° C. using an ice-bath. Then 2.30 mL methanesulfonyl chloride (3.40 g, 29.7 mmol, 2.5 eq.), followed by 4.04 mL diisopropylethylamine (2.40 g, 23.76 mmol, 2 eq.) and stirring was continued at 0° C. for 1 h.

Then the reaction mixture was diluted with 25 mL DCM and washed with 40 mL half-saturated brine. The aqueous layer was extracted once with 10 mL DCM. The combined organic layers were dried over Na₂SO₄, filtered and concentrated under reduced pressure to give 3.39 g of (3S)-1-(bicyclo[22.1]hept-5-ene-2-carbonyl)piperidin-3-yl methanesulfonate.

### Example 2a: ((R)-3-(4-amino-3-(4-phenoxyphenyl)-1H-pyrazolo[3,4-d]pyrimidin-1-yl)piperidin-1-yl)(bicyclo[2.2.1]hept-5-en-2-yl)methanone

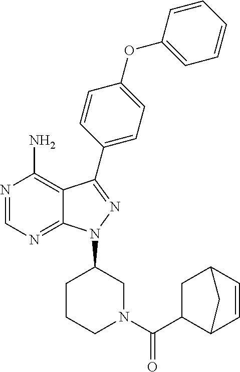 [SMILES: Nc1ncnc2c1c(-c1ccc(Oc3ccccc3)cc1)nn2[C@@H]1CCCN(C(=O)C2CC3C=CC2C3)C1]

78 mg 3-(4-phenoxyphenyl)-1H-pyrazolo[3,4-d]pyrimidin-4-amine (0.25 mmol, 1 eq.) (synthesized following WO 0119829) and 77 mg of (3S)-1-(bicyclo[2.2.1]hept-5-ene-2-carbonyl)piperidin-3-yl methanesulfonate (0.25 mmol, 1 eq.) were dissolved in 3 mL of DMF.

Then 44 mg NaOEt (0.65 mmol, 2.5 eq.) were added and the reaction mixture was stirred at 80° C. overnight.

The residue was dissolved in ethyl acetate and washed twice with water then brine, dried over Na₂SO₄, filtered and concentrated in vacuo. The residue was loaded on a silica column and the product eluted using ethyl acetate. The product fractions were then concentrated to give 10 mg of product.

¹H NMR (500 MHz, DMSO-d₆): 8.47, 8.26 (s, 1H), 7.67 (m, 2H), 7.43 (t, J=7.9 Hz, 2H), 7.19 (t, 7.3 Hz, 1H), 7.16 (d, 8.7 Hz, 2H), 7.13 (d, 8.4 Hz, 2H), 6.19-5.95 (m, 2H), 4.83-4.62 (m, 1H), 4.52 (dd, J=12.3, 3.4 Hz, 0.25H), 4.43 (dd, J=12.7, 3.7 Hz, 0.25H), 4.11 (dd, J=13.6, 3.5 Hz, 0.25H), 4.05 (dd, J=13.7, 3.4 Hz, 0.25H), 3.88 (bd, J=13.4 Hz, 0.5H), 3.86 (bd, J=13.9 Hz, 0.25H), 3.60 (dd, J=13.3, 9.8 Hz, 0.25H), 3.52 (dd, J=13.1, 10.0 Hz, 0.25H), 3.25 (dd, J=12.7, 10.0 Hz, 0.5H), 3.18 (m, 1H), 3.0-2.7 (m, 2H), 2.39 (dd, J=8.6, 4.4 Hz, 0.5H), 2.35-2.2 (m, 1.5H), 2.13 (m, 1H), 1.98-1.88 (m, 1H), 1.83 (m, 0.5H), 1.8-1.5 (m, 1.5H), 1.5-1.3 (m, 2H), 1.24 (m, 1H).

### Example 2b: ((R)-3-(4-amino-3-(4-phenoxyphenyl)-1H-pyrazolo[3,4-d]pyrimidin-1-yl)piperidin-1-yl)(bicyclo[2.2.1]hept-5-en-2-yl)methanone

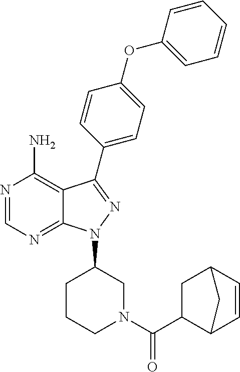 [SMILES: Nc1ncnc2c1c(-c1ccc(Oc3ccccc3)cc1)nn2[C@@H]1CCCN(C(=O)C2CC3C=CC2C3)C1]

230 mg 3-(4-phenoxyphenyl)-1H-pyrazolo[3,4-d]pyrimidin-4-amine (0.76 mmol, 1 eq.) and 500 mg of (3S)-1-(bicyclo[2.2.1]hept-5-ene-2-carbonyl)piperidin-3-yl methane-sulfonate (1.67 mmol, 2.2 eq.) were dissolved in 8 mL of methyl isobutyl ketone (MIBK). Then 4 mL of sat. K₂CO₃-solution and 0.24 mL phase transfer catalyst Aliquat® 175 (75% in water) were added. The biphasic mixture was then heated to 94° C. and stirred under reflux overnight. The mixture was then cooled to 30° C. and the aqueous layer separated. The organic layer was washed once with water and then concentrated in vacuo.

The residue was dissolved in ethyl acetate and washed twice with water then brine, dried over Na₂SO₄, filtered and concentrated in vacuo. The residue was loaded on a silica column and the product eluted using heptane: ethyl acetate 1:1. The product fractions were then concentrated to give 47 mg of product.

### Example 2c: ((R)-3-(4-amino-3-(4-phenoxyphenyl)-1H-pyrazolo[3,4-d]pyrimidin-1-yl)piperidin-1-yl)((2R)-bicyclo[2.2.1]hept-5-en-2-yl)methanone

2.92 g (R)-3-(4-phenoxyphenyl)-1-(piperidin-3-yl)-1H-pyrazolo[3,4-d]pyrimidin-4-amine (6.9 mmol, 1.2 eq.), 2.21 g 1-ethyl-3-(3-dimethylaminopropyl)carbodiimid hydrochloride (EDC-HCl) (11.5 mmol, 2 eq) and 0.78 g 1-hydroxybenzotriazol (HOBt) (5.8 mmol, 1 eq.) were dissolved in 50 mL DMF and 2.4 mL triethylamine (1.75 g, 17.3 mmol, 3 eq.) were added. Then 0.795 g exo-5-norbornene-2-carboxylic acid (5.8 mmol, 1 eq.) were added and the reaction mixture stirred at room temperature for 3 h.

The mixture was then poured on 50 mL H₂O and 100 mL ethyl acetate. The pH was then adjusted to 1.3 using 6M HCl. After separation of the organic layer, the aqueous layer was extracted with 50 mL ethyl acetate. The combined organic layers were washed subsequently with 50 mL 0.5M NaOH and 50 mL H₂O and 50 mL half-saturated brine, dried with Na₂SO₄, filtered and concentrated in vacuo to give 2.25 g of ((R)-3-(4-amino-3-(4-phenoxyphenyl)-1H-pyrazolo[3,4-d]pyrimidin-1-yl)piperidin-1-yl)(bicyclo[2.2.1]hept-5-en-2-yl)methanone as a clear oil.

### Example 3

Ibrutinib (I)

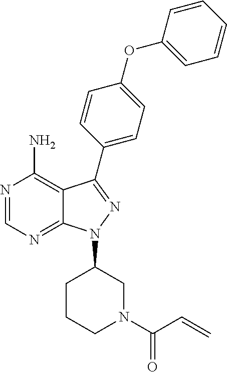 [SMILES: C=CC(=O)N1CCC[C@@H](n2nc(-c3ccc(Oc4ccccc4)cc3)c3c(N)ncnc32)C1]

30 mg ((R)-3-(4-amino-3-(4-phenoxyphenyl)-1H-pyrazolo[3,4-d]pyrimidin-1-yl)piperidin-1-yl)(bicyclo[2.2.1]hept-5-en-2-yl)methanone were dissolved in 1.5 mL diphenyl ether. The solution was then heated to 250° C. for 10 min. Then the solvent was removed under reduced pressure.

The residue was then triturated twice with heptane and once with diethyl ether to give 20 mg of Ibrutib.

¹H NMR (500 MHz, DMSO-d₆): 8.27 (s, 1H), 7.67 (m, 2H), 7.44 (t, J=7.9 Hz, 2H), 7.19 (t, J=7.3 Hz, 1H), 7.16 (d, J=8.5 Hz, 2H), 7.13 (d, J=8.1 Hz, 2H), 6.87 (dd, J=16.3, 10.6 Hz, 0.5H), 6.72 (dd, J=16.1, 10.8 Hz, 0.5H), 6.15 (d, J=16.5, Hz, 0.5H), 6.07 (d, J=16.5, Hz, 0.5H), 5.71 (d, J=10.1, Hz, 0.5H), 5.60 (d, J=10.1, Hz, 0.5H), 4.71 (m, 1H), 4.57 (d, J=11.2 Hz, 0.5H), 4.22 (m, 1H), 4.08 (d, J=13.4 Hz, 0.5H), 3.72 (dd, J=12.3, 10.5 Hz, 0.5H), 3.21 (m, 1H), 3.02 (dd, J=12.0, 10.9 Hz, 0.5H), 2.29 (m, 1H), 2.14 (m, 1H), 1.92 (m, 1H), 1.60 (m, 1H).

### Example 4a

100 g (S)-tert-butyl 3-hydroxypiperidine-1-carboxylate (497 mmol) was dissolved in 500 mL anisole and the resulting mixture cooled to 0° C. Then 63.2 g methanesulfonyl chloride (42.7 mL, 551 mmol) was added. Then 55.9 g triethylamine (77 mL, 552 mmol) was added dropwise within one hour, keeping the reaction temperature below 5° C. (exothermic addition); afterwards, the reaction mixture was stirred for additional 30 min at 5° C. Then, the mixture is warmed to 15° C. followed by addition of a 380 mL half-saturated (13% w/w) aqueous NaCl—solution. The lower aqueous layer is removed and the organic layer is washed with 380 mL half-saturated aqueous NaCl—solution. After final removal of the lower aqueous layer, the anisole phase containing (S)-tert-butyl 3-((methylsulfonyl)oxy)piperidine-1-carboxylate is used for the next chemical step.

In a second reaction vessel, 172 g K₂CO₃ (1244 mmol) and 12 g tetrabutyl ammoniumbromide (TBAB) (37 mmol) are suspended in 200 mL anisole. After addition of 75.4 g 3-(4-phenoxyphenyl)-1H-pyrazolo[3,4-d]pyrimidin-4-amine (248 mmol), the solution of the mesylate in anisole (obtained in the first step) is transferred to the reaction vessel. The heterogeneous mixture is then heated to 117° C. until >95% conversion is observed by HPLC—typically after 24 h. The reaction mixture is then cooled to 80° C. and 600 mL H₂O is added. After stirring for 15 minutes, the lower, aqueous layer is separated at ˜50° C.

To the organic layer 123 g conc. HCl (37%, 104 mL, 1248 mmol) was added and the mixture warmed to 70° C. and stirred for 1 h. Then the reaction mixture is cooled to 50° C. and the lower, aqueous layer is separated. After washing of the organic is layer with 10 mL of H₂O, the organic layer is discarded and the combined aqueous layers are added slowly to 2.1 L isopropanol heated to reflux. After stirring for 2 h, the crystal suspension is cooled to 0° C., stirred for another 2 h. The product is isolated by filtration by a vacuum nutsche, followed by washing with 800 mL cold (−20° C.) isopropanol and then dried in vacuo to give 86.5 g of (R)-3-(4-amino-3-(4-phenoxyphenyl)-1H-pyrazolo[3,4-d]pyrimidin-1-yl)piperidin-1-ium dihydrochloride salt. e.r. (HPLC)=96.1:3.9

### Example 4b

The reaction was carried out as described in Example 4a, but wherein processing of the reaction mixture was as follows:

To the organic layer 105 g conc. HCl (37%, 89 mL, 1066 mmol) was added and the mixture warmed to 70° C. and stirred for 1 h. Then the reaction mixture is cooled to 50° C. and 181 mL of isopropanol are added. The mixture is then cooled to 18° C. and the pH is adjusted to 4.7 by addition of 54 mL triethylamine. After stirring for 5 h, the crystal suspension is cooled to −10° C. within 5 h and stirred for another 10 h at −10° C. The product is isolated by filtration by a vacuum nutsche, followed by washing with 800 mL cold (−20° C.) isopropanol and then dried in vacuo to give 68.2 g of (R)-3-(4-amino-3-(4-phenoxyphenyl)-1H-pyrazolo[3,4-d]pyrimidin-1-yl)piperidin-1-ium monohydrochloride salt. e.r. (HPLC)=99.9:0.1

### Example 5

125 g (S)-tert-butyl 3-hydroxypiperidine-1-carboxylate (448 mmol) was dissolved in 1.25 L MED and the mixture cooled to 0° C. Then 78.3 g methanesulfonyl chloride (52.9 mL, 683 mmol) was added, followed by slow addition of 69.1 g triethylamine (95.2 mL, 683 mmol), keeping the temperature below 5° C. After stirring for 1 h, the reaction is quenched by addition of 780 mL half-saturated (13% w/w) NaCl solution. After warming to room temperature, the layers are separated; the organic layer is washed once with 220 mL 780 mL half-saturated (13% w/w) NaCl solution and then concentrated in vacuo to approx. 260 mL. At 50° C. mass temperature, 1.4 L isopropanol is added, the mixture is concentrated to a volume of 930 mL. This step is repeated until a residual amount of dichloromethane <0.5 area %, as determined by GC. Then the mixture is stirred at room temperature and 100 mg of seeding crystals are added. After stirring for 1 h, the mixture is cooled to −20° C. and stirring continued for 1 h. The product is then isolated by filtration by a vacuum nutsche, followed by washing with a total of 300 mL cold (−20° C.) isopropanol and then dried in vacuo to give 159 g (S)-tert-butyl 3-((methylsulfonyl)oxy)piperidine-1-carboxylate.

592 g K₂CO₃ (4283 mmol) and 74 g tetrabutyl ammoniumbromide (TBAB) (229 mmol) are dissolved in 620 mL H₂O. Then, 1.2 L MIBK and 86.9 g 3-(4-phenoxyphenyl)-1H-pyrazolo[3,4-d]pyrimidin-4-amine (286 mmol) are added. The mixture is warmed to 70° C., when 159 g (S)-tert-butyl 3-((methylsulfonyl)oxy)piperidine-1-carboxylate (886 mmol) is added. The mixture is then heated to reflux is and stirred overnight, until >90% conversion is observed by HPLC. The reaction mixture is then cooled to 40° C. and 200 mL H₂O is added. After stirring for 15 minutes, the lower, aqueous layer is separated and the organic layer is washed twice with 800 mL of H₂O.

The organic layer was then concentrated in vacuo and the residue re-dissolved in 1.9 L of isopropanol at 50° C. Then 154 g conc. HCl (37%, 131 mL, 1329 mmol) was added and the mixture warmed to 70° C. and stirred for 1 h. Then the reaction mixture is cooled to 50° C. and seeding crystals are added. After stirring for 1 h, the crystal suspension is cooled to 0° C., stirred for another 2 h. The product is isolated by filtration by a vacuum nutsche, followed by washing with 400 mL cold (−20° C.) isopropanol and then dried in vacuo to give 100 g of (R)-3-(4-amino-3-(4-phenoxyphenyl)-1H-pyrazolo[3,4-d]pyrimidin-1-yl)piperidin-1-ium dihydrochloride salt. e.r. (HPLC)=98.3:1.7 In summary, the present invention refers to the following preferred embodiments:

1. A process for the preparation of a compound of formula (IV),

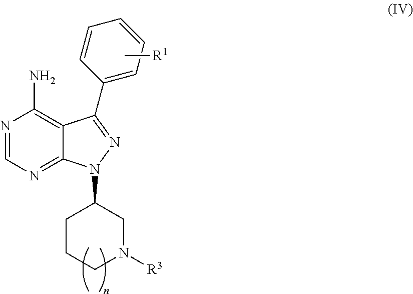

comprising the steps of:

(a) reacting a compound of formula (II)

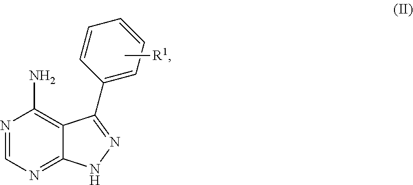

in the presence of an alkaline substance, and optionally in the presence of a phase transfer catalyst, with a compound of formula (III)

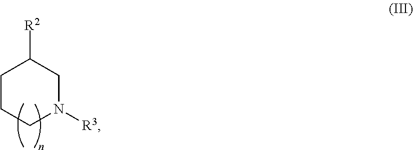

to obtain a compound of formula (IV),
wherein R is selected from substituted or non-substituted alkyl, substituted or non-substituted heteroalkyl, substituted or non-substituted cycloalkyl, substituted or non-substituted heterocycloalkyl, OR⁴, SR⁴, NR⁴R⁵, and halogen,
R⁴ and R⁵ are individually selected from hydrogen, substituted or non-substituted alkyl, substituted or non-substituted heteroalkyl, substituted or non-substituted cycloalkyl, substituted or non-substituted heterocycloalkyl, substituted or non-substituted aryl, and substituted or non-substituted heteroaryl,
n is 0 to 3,
R² is a leaving group,
R³ is selected from hydrogen, a group selected from carbamoyl, carbamates of the formula C(O)O—R⁹, substituted or non-substituted benzyl and substituted or non-substituted silyl, and C(O)—R⁶, wherein
R⁶ is selected from hydrogen, substituted or non-substituted alkyl, substituted or non-substituted heteroalkyl, substituted or non-substituted cycloalkyl, substituted or non-substituted heterocycloalkyl, substituted or non-substituted alkenyl, substituted or non-substituted cycloalkenyl, substituted or non-substituted heterocycloalkenyl substituted or non-substituted aryl, and substituted or non-substituted heteroaryl, and
R⁹ is selected from substituted or non-substituted alkyl, substituted or non-substituted heteroalkyl, substituted or non-substituted cycloalkyl, substituted or non-substituted heterocycloalkyl, substituted or non-substituted alkenyl, substituted or non-substituted cycloalkenyl, substituted or non-substituted heterocycloalkenyl substituted or non-substituted aryl, and substituted or non-substituted heteroaryl.

2. The process of embodiment 1, wherein the phase transfer catalyst is a compound of the formula

NR⁸₄⁺X⁻ or PR⁸₄⁺X⁻
wherein R⁸ is individually selected from substituted or non-substituted alkyl, substituted or non-substituted heteroalkyl, substituted or non-substituted cycloalkyl, substituted or non-substituted heterocycloalkyl, substituted or unsubstituted aryl or substituted or unsubstituted heteroaryl and
X is selected from Cl, Br, F, SbF₆, PF₆, BF₄, ClO₄, HSO₄, HCO₃, NO₃, CF₃COO, alkyl-COO, aryl-COO, alkyl-SO₃, aryl-SO₃, and CF₃SO₃.

3. The process of embodiment 1 or 2, wherein R³ is C(O)—R⁶, and R⁶ is a group selected from

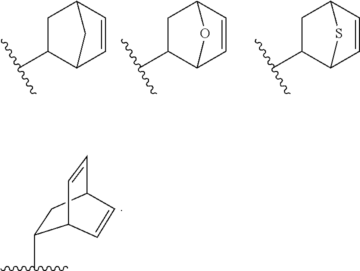

4. The process of anyone of embodiments 1 to 3, wherein the compound of formula (III) is a compound having the formula (IIIa)

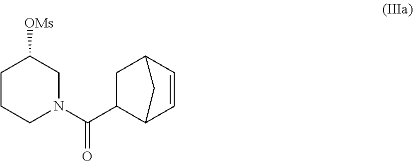 [SMILES: CS(=O)(=O)O[C@H]1CCCN(C(=O)C2CC3C=CC2C3)C1]

5. A process for the preparation of a compound of formula (I),

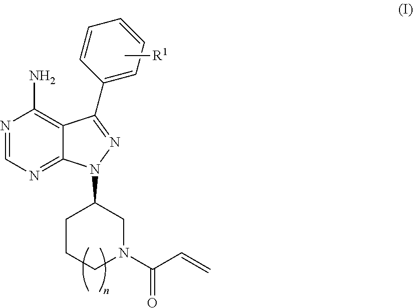

by subjecting a compound of formula (IV)

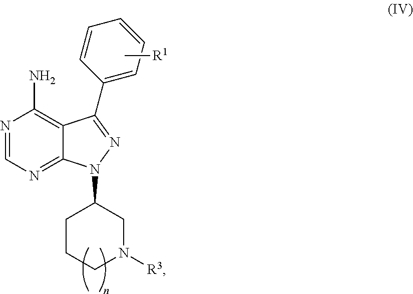

to deprotection to obtain the compound of formula (I),
wherein R is selected from substituted or non-substituted alkyl, substituted or non-substituted heteroalkyl, substituted or non-substituted cycloalkyl, substituted or non-substituted heterocycloalkyl, OR⁴, SR, NR⁴R⁵, and halogen,
R⁴ and R⁵ are individually selected from hydrogen, substituted or non-substituted alkyl, substituted or non-substituted heteroalkyl, substituted or non-substituted cycloalkyl, substituted or non-substituted heterocycloalkyl, substituted or non-substituted aryl, and substituted or non-substituted heteroaryl,
n is 0 to 3,
R³ is C(O)—R⁶, and

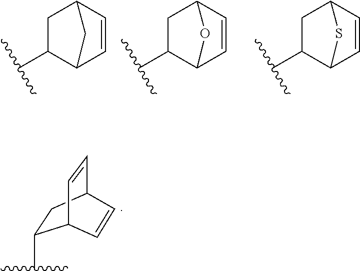

R⁶ is a group selected from

6. The process of embodiment 5, wherein R³ is C(O)—R⁶, and R⁶ is

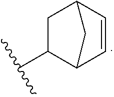 [SMILES: *C1CC2C=CC1C2]

7. The process of embodiment 5 or 6, wherein
R¹ is OPhenyl,
n is 1, and
R³ is C(O)—R⁶, and R⁶ is

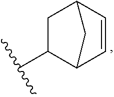 [SMILES: *C1CC2C=CC1C2]

to obtain a compound of formula (Ia)

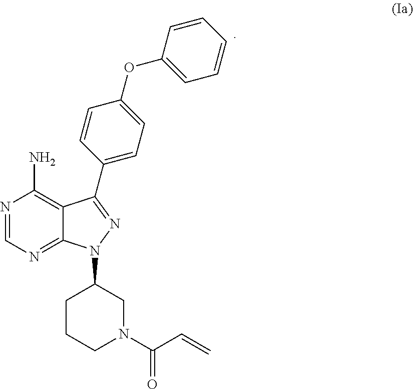 [SMILES: C=CC(=O)N1CCC[C@@H](n2nc(-c3ccc(Oc4ccccc4)cc3)c3c(N)ncnc32)C1]

8. The process of any one of embodiments 5 to 7, wherein the compound of formula (IV) is prepared by the process of any one of claims 1 to 4.

9. A process for the preparation of a compound of formula (Ia),

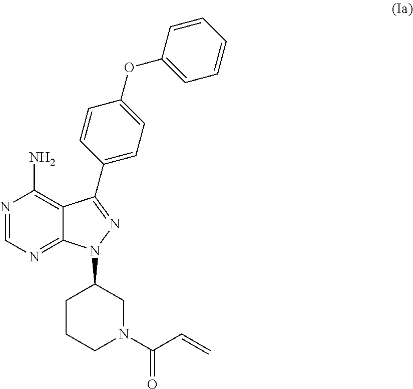 [SMILES: C=CC(=O)N1CCC[C@@H](n2nc(-c3ccc(Oc4ccccc4)cc3)c3c(N)ncnc32)C1]

comprising the process steps of:

(a) reacting a compound of formula (Ia)

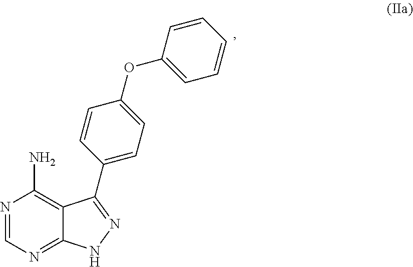 [SMILES: Nc1ncnc2[nH]nc(-c3ccc(Oc4ccccc4)cc3)c12]

in the presence of an alkaline substance, and optionally in the presence of a phase transfer catalyst, with a compound of formula (IIa)

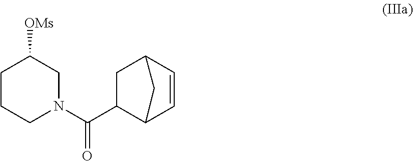 [SMILES: CS(=O)(=O)O[C@H]1CCCN(C(=O)C2CC3C=CC2C3)C1]

to obtain a compound of formula (IVa),

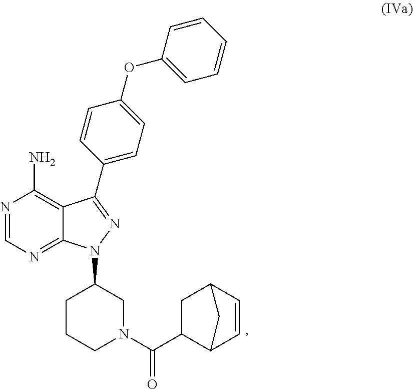 [SMILES: Nc1ncnc2c1c(-c1ccc(Oc3ccccc3)cc1)nn2[C@@H]1CCCN(C(=O)C2CC3C=CC2C3)C1]

and subjecting a compound of formula (IVa) to deprotection to obtain the compound of formula (Ia).

10. A process for the preparation of a compound of formula (VIII),

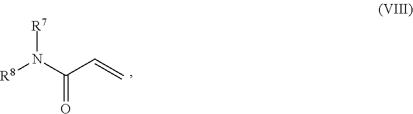

comprising the steps of:

subjecting a compound of formula (VII)

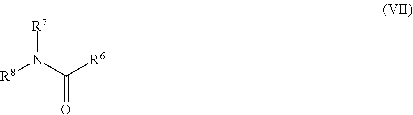

to deprotection,
wherein R⁶ is a group selected from

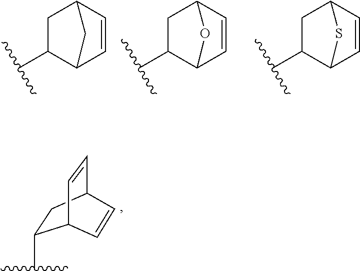

and
wherein R⁷ and R⁸ are individually selected from hydrogen, substituted or non-substituted alkyl, substituted or non-substituted heteroalkyl, substituted or non-substituted cycloalkyl, substituted or non-substituted heterocycloalkyl, substituted or non-substituted aryl, and substituted or non-substituted heteroaryl.

11. The process of embodiment 10, wherein R⁷ and R⁸ together form a cyclic structure selected from substituted or non-substituted cycloalkyl and substituted or non-substituted heterocycloalkyl.

12. A composition comprising a compound of formula (Ia) and a compound of formula (Va)

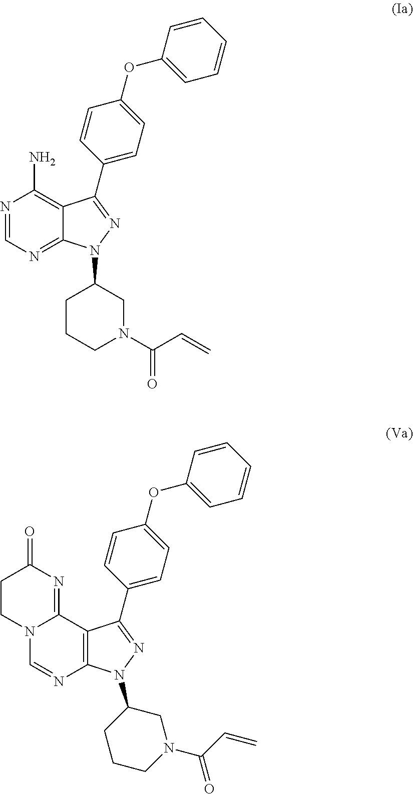 [SMILES: C=CC(=O)N1CCC[C@@H](n2nc(-c3ccc(Oc4ccccc4)cc3)c3c(N)ncnc32)C1 ; C=CC(=O)N1CCC[C@@H](n2nc(-c3ccc(Oc4ccccc4)cc3)c3c2N=CN2CCC(=O)N=C32)C1]

13. A compound represented by the formula (IIIa)

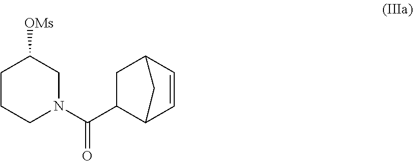 [SMILES: CS(=O)(=O)O[C@H]1CCCN(C(=O)C2CC3C=CC2C3)C1]

14. A compound represented by the formula (IVb)

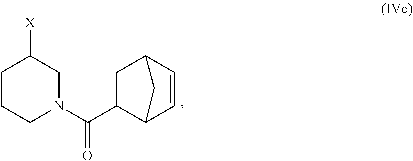

wherein X is a N-linked heterocycle.

15. A compound represented by the formula (Vb),

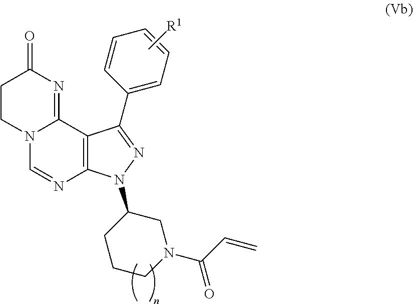

wherein n is 0 to 3, preferably is 1, and R¹ is OPhenyl.

1. A process for the preparation of a compound of formula (IV), 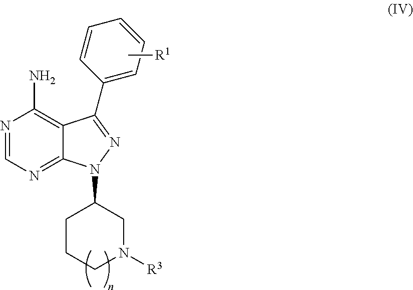
comprising the steps of:
(a) reacting a compound of formula (II) 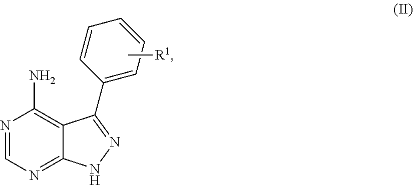
in the presence of an alkaline substance, and optionally in the presence of a phase transfer catalyst, with a compound of formula (III) 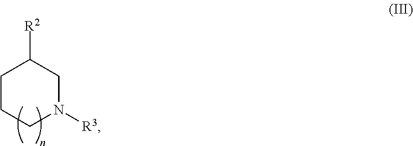
to obtain a compound of formula (IV),
wherein R¹ is selected from substituted or non-substituted alkyl, substituted or non-substituted heteroalkyl, substituted or non-substituted cycloalkyl, substituted or non-substituted heterocycloalkyl, OR⁴, SR⁴, NR⁴R⁵, and halogen,
R⁴ and R⁵ are individually selected from hydrogen, substituted or non-substituted alkyl, substituted or non-substituted heteroalkyl, substituted or non-substituted cycloalkyl, substituted or non-substituted heterocycloalkyl, substituted or non-substituted aryl, and substituted or non-substituted heteroaryl,
n is 0 to 3,
R² is a leaving group,
R³ is selected from hydrogen, a group selected from carbamoyl, carbamates of the formula C(O)O—R⁹, substituted or non-substituted benzyl and substituted or non-substituted silyl, and C(O)—R⁶, wherein
R⁶ is selected from hydrogen, substituted or non-substituted alkyl, substituted or non-substituted heteroalkyl, substituted or non-substituted cycloalkyl, substituted or non-substituted heterocycloalkyl, substituted or non-substituted alkenyl, substituted or non-substituted cycloalkenyl, substituted or non-substituted heterocycloalkenyl substituted or non-substituted aryl, and substituted or non-substituted heteroaryl, and
R⁹ is selected from substituted or non-substituted alkyl, substituted or non-substituted heteroalkyl, substituted or non-substituted cycloalkyl, substituted or non-substituted heterocycloalkyl, substituted or non-substituted alkenyl, substituted or non-substituted cycloalkenyl, substituted or non-substituted heterocycloalkenyl substituted or non-substituted aryl, and substituted or non-substituted heteroaryl.

2. The process of claim 1, wherein the phase transfer catalyst is a compound of the formula
NR⁸₄⁺X⁻ or PR⁸₄⁺X⁻
wherein R⁸ is individually selected from substituted or non-substituted alkyl, substituted or non-substituted heteroalkyl, substituted or non-substituted cycloalkyl, substituted or non-substituted heterocycloalkyl, substituted or unsubstituted aryl or substituted or unsubstituted heteroaryl and
X is selected from Cl, Br, F, SbF₆, PF₆, BF₄, ClO₄, HSO₄, HCO₃, NO₃, CF₃COO, alkyl-COO, aryl-COO, alkyl-SO₃, aryl-SO₃, and CF₃SO₃.

3. The process of claim 1, wherein R³ is C(O)—R⁶, and
R⁶ is a group selected from

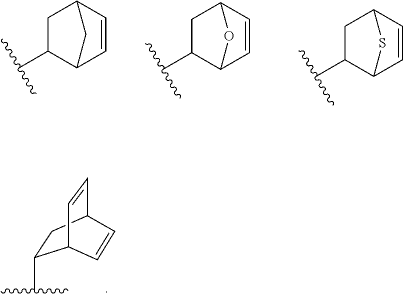 [SMILES: *C1CC2C=CC1C2 ; *C1CC2C=CC1O2 ; *C1CC2C=CC1S2 ; *C1CC2C=CC1C=C2]

4. The process of claim 1, wherein the compound of formula (III) is a compound having the formula (IIIa)

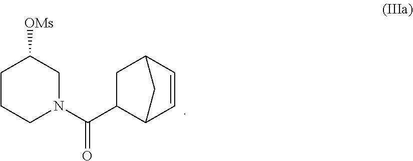 [SMILES: CS(=O)(=O)O[C@H]1CCCN(C(=O)C2CC3C=CC2C3)C1]

5. A process for the preparation of a compound of formula (I), 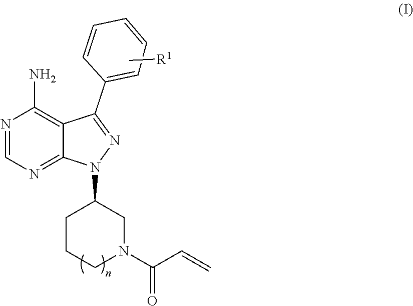
by subjecting a compound of formula (IV), which is prepared by the process according to claim 1, 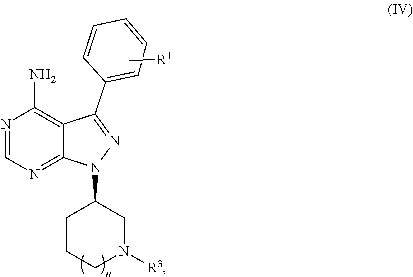
to deprotection to obtain the compound of formula (I),
wherein R¹ is selected from substituted or non-substituted alkyl, substituted or non-substituted heteroalkyl, substituted or non-substituted cycloalkyl, substituted or non-substituted heterocycloalkyl, OR⁴, SR⁴, NR⁴R⁵, and halogen,
R⁴ and R⁵ are individually selected from hydrogen, substituted or non-substituted alkyl, substituted or non-substituted heteroalkyl, substituted or non-substituted cycloalkyl, substituted or non-substituted heterocycloalkyl, substituted or non-substituted aryl, and substituted or non-substituted heteroaryl,
n is 0 to 3,
R³ is C(O)—R⁶, and
R⁶ is a group selected from

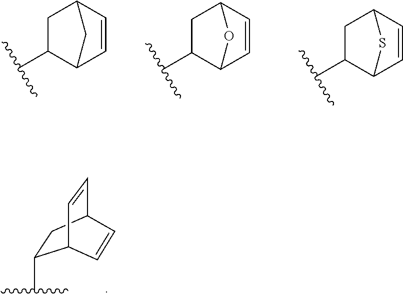 [SMILES: *C1CC2C=CC1C2 ; *C1CC2C=CC1O2 ; *C1CC2C=CC1S2 ; *C1CC2C=CC1C=C2]

6. The process of claim 5, wherein R³ is C(O)—R⁶, and R⁶ is

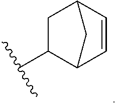 [SMILES: *C1CC2C=CC1C2]

7. The process of claim 5, wherein
R¹ is OPhenyl,
n is 1, and
R³ is C(O)—R⁶, and R⁶ is 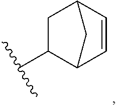 [SMILES: *C1CC2C=CC1C2]
to obtain a compound of formula (Ia)

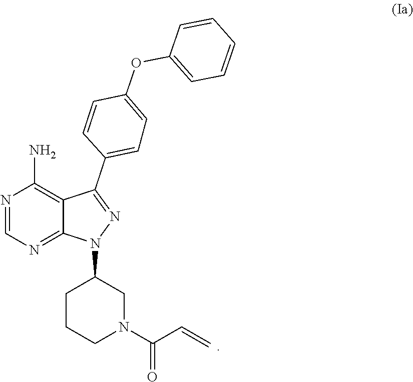 [SMILES: C=CC(=O)N1CCC[C@@H](n2nc(-c3ccc(Oc4ccccc4)cc3)c3c(N)ncnc32)C1]

8. A process for the preparation of a compound of formula (Ia), 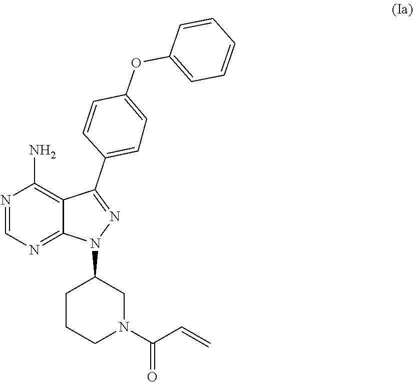 [SMILES: C=CC(=O)N1CCC[C@@H](n2nc(-c3ccc(Oc4ccccc4)cc3)c3c(N)ncnc32)C1]
comprising the process steps of:
(a) reacting a compound of formula (IIa) 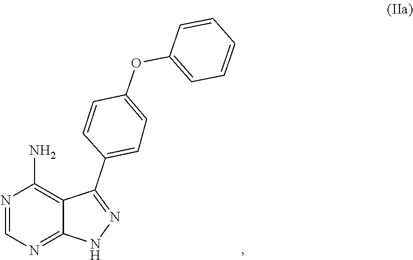 [SMILES: Nc1ncnc2[nH]nc(-c3ccc(Oc4ccccc4)cc3)c12]
in the presence of an alkaline substance, and optionally in the presence of a phase transfer catalyst, with a compound of formula (IIa) 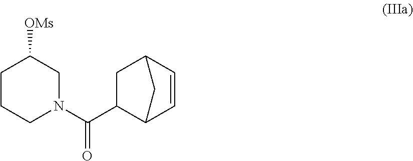 [SMILES: CS(=O)(=O)O[C@H]1CCCN(C(=O)C2CC3C=CC2C3)C1]
to obtain a compound of formula (IVa), 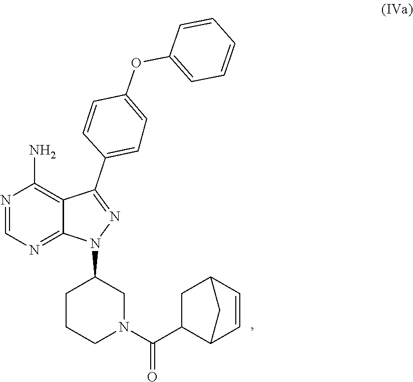 [SMILES: Nc1ncnc2c1c(-c1ccc(Oc3ccccc3)cc1)nn2[C@@H]1CCCN(C(=O)C2CC3C=CC2C3)C1]
and subjecting a compound of formula (IVa) to deprotection to obtain the compound of formula (Ia).

9. A composition comprising a compound of formula (Ia) and a compound of formula (Va) 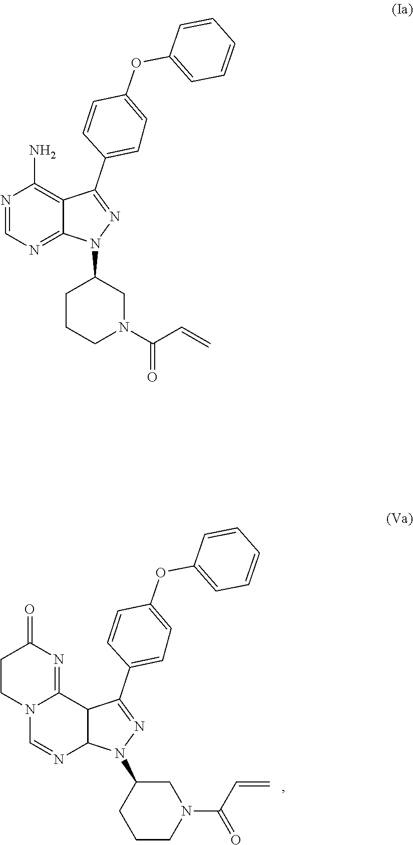 [SMILES: C=CC(=O)N1CCC[C@@H](n2nc(-c3ccc(Oc4ccccc4)cc3)c3c(N)ncnc32)C1 ; C=CC(=O)N1CCC[C@@H](N2N=C(c3ccc(Oc4ccccc4)cc3)C3C4=NC(=O)CCN4C=NC32)C1]
wherein the compound of formula (Ia) is prepared by the process according to claim 8, and
wherein the amount of the compound of formula (Va) is 0.5 wt. % or less.

10. A compound represented by the formula (IIIa)

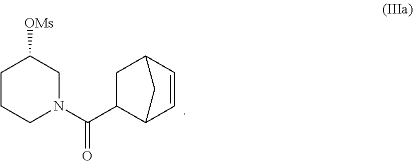 [SMILES: CS(=O)(=O)O[C@H]1CCCN(C(=O)C2CC3C=CC2C3)C1]

11. A compound represented by the formula (Vb), 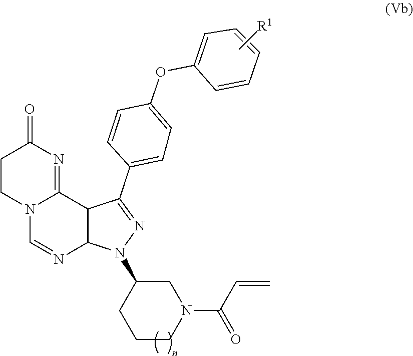
wherein n is 0 to 3, and R¹ is OPhenyl.
